(19) United States
(12) Patent Application Publication
(10) Pub. No.: US 2009/0171424 A1
(43) Pub. Date: Jul. 2, 2009

(54) RF DEVICE FOR HEATING BIOLOGICAL TISSUE USING A VIBRATING APPLICATOR

(75) Inventors: Alexander BRITVA, Migdal Haemek (IL); Ziv Karni, Kfar Shmaryahu (IL)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(73) Assignee: ALMA LASERS LTD., Caesarea (IL)

(21) Appl. No.: 11/964,759

(22) Filed: Dec. 27, 2007

(51) Int. Cl. A61B 18/18

(52) U.S. Cl. 607/101

(57) ABSTRACT

Apparatus and methods for treating biological tissue 200 with RF power delivered from an applicator 240, at least a portion of which mechanically vibrates, are disclosed. In some embodiments, the presently disclosed apparatus includes a vibration generation device 190 operative to cause the at least a portion of the applicator 240 to mechanically vibrate. Typically, the mechanical vibrations have a frequency of between 1 Hz and 100 Hz, and an amplitude of between 0.1-10 mm. In some embodiments, the vibrations primarily include vibrations in a direction substantially perpendicular to a surface of the biological tissue 200 in contact with the applicator 240 through which RF power is delivered. In some embodiments, the vibration parameters (i.e. amplitude or frequency) are determined in accordance with one or more physical parameters associated with the delivering of the RF power to the biological tissue.

### FIELD OF THE INVENTION

The present invention relates to apparatus and methods for heating biological tissue using RF energy.

### BACKGROUND OF THE INVENTION

The following published documents are believed to represent the current state of the art and the contents thereof are hereby incorporated by reference: US 2007/0106349, U.S. Pat. No. 5,755,753, U.S. Pat. No. 7,241,291, and WO/2007/117580.

### SUMMARY OF THE INVENTION

The present inventors are now disclosing that when treating biological tissue (for example, skin tissue) with RF power, it is useful to do so using an applicator which mechanically vibrates when RF power is applied. In some embodiments, at least a portion of the applicator (for example, a portion in contact with an upper surface of the tissue) vibrates in a direction that is substantially perpendicular to an upper surface of the biological tissue.

In one non-limiting scenario, the device is used as follows: (i) the “vibrating RF applicator” of the presently-disclosed device is placed in contact with the skin surface; (ii) RF-power is delivered from the applicator to the skin, thereby heating, for example, underlying tissue layers; (iii) concomitant with the delivery of RF-power, at least a portion of the applicator, for example a skin-contacting portion of the applicator, is caused to mechanically vibrate.

In one non-limiting example, the presently disclosed device and treatment methods are useful for heating and contraction of adipose tissues and/or as a means of cellulite reduction. Thus, in one non-limiting example, the present inventor contemplates modifying a device similar to that disclosed in US 2007/0106369 (for example, a device including any combination or sub-combination of features disclosed in US 2007/0106369) to include a mechanical vibration generation device configured to cause a portion of the applicator to mechanically vibrate.

In yet another non-limiting example, the presently disclosed device and methods are useful for applications related to collagen restructuring and/or wrinkle treatment.

It is now disclosed for the first time an apparatus for treatment of a biological tissue of a subject comprising: a) an applicator contactable with a surface of the tissue; b) an RF power source configured to produce at least 20 Watts of REF power directed to the applicator; and c) a vibration generation device mechanically linked to the applicator, the vibration generation device being operative to generate mechanical vibrations of at least a portion of the applicator including mechanical vibrations having a frequency of at least 1 Hz and at most 100 Hz.

According to some embodiments, the apparatus further comprises a phase shifter operative to control a phase of electromagnetic wave carried the RF-power.

According to some embodiments, the apparatus further comprises an impedance matching network (IMN) operative to match an impedance power source to impedance of the biological tissue.

According to some embodiments, the apparatus further comprises an RF resonator connected to the applicator, the RF resonator operative to cyclically accumulate and release a desired amount of RF energy.

According to some embodiments, the applicator includes only a single electrode with a dielectric barrier associated with an outside surface of the applicator.

According to some embodiments, the applicator is made primarily from electrically conductive materials.

According to some embodiments, the RF power source, the applicator and the vibration generation device are configured so that, when the applicator is contacted to the surface of the biological tissue: i) the applicator is operative to deliver the RF power to the contacted biological tissue; ii) the vibration generation device is operative such that the mechanical vibrations of the at least a portion of the applicator include vibrations in a direction that is substantially parallel to a wavefront propagation direction of the RF power delivered from the applicator to the biological tissue.

According to some embodiments, the RF power source, the applicator and the vibration generation device are configured so that, when the applicator is contacted to the surface of the biological tissue: i) the applicator is operative to deliver the RF power to the contacted biological tissue via an applicator contact region of the applicator; ii) the vibration generation device and the applicator are operative such that an average direction of generated mechanical vibrations at the applicator contact region is substantially parallel to a wavefront propagation direction of the RF power delivered from the applicator to the biological tissue.

According to some embodiments, the vibration generation device and the applicator are configured such that the generated mechanical vibrations of the at least a portion include mechanical vibrations having a frequency of at least 2 Hz and at most 10 Hz.

According to some embodiments, the vibration generation device and the applicator are configured such that the generated mechanical vibrations of the at least a portion include mechanical vibrations having an amplitude of at least 0.1 mm and more. In different embodiments, the amplitude may be at least 0.2 mm, 0.3 mm, 0.5 mm or 1 mm.

According to some embodiments, the vibration generation device and the applicator are configured such that the generated mechanical vibrations of the at least a portion include mechanical vibrations having an amplitude of at most 10 mm.

According to some embodiments, the vibration generation device includes a linearly oscillating mass.

According to some embodiments, the vibration generation device includes: i) a rotary motor; and ii) a rotary-to-linear motion converter operatively linked to the motor.

According to some embodiments, the vibration generation device is embedded within the applicator.

According to some embodiments, the vibration generation device is operative to generate remote vibrations remote to the applicator and the apparatus further comprises: a vibration transmitter operative to transmit the remote vibration to the applicator. Exemplary mechanisms for transmitting the remote vibration include but are not limited to hydraulic mechanisms and pneumatic mechanisms.

According to some embodiments, the vibration generation device includes at least one of: i) an electromagnetic actuator; ii) a piezoelectric actuator; and iii) a magnetostrictive actuator.

According to some embodiments, i) the apparatus further comprises a tissue softness detector operative to detect a softness of the biological tissue contacted by the applicator and ii) the vibration generation device includes a vibration controller operative to provide at least one of a vibration frequency and a vibration amplitude in accordance with results of the tissue softness detecting.

According to some embodiments, the vibration controller is operative to provide in increased frequency contingent on detecting increased tissue softness.

According to some embodiments, i) the apparatus further comprises a applicator movement speed detector operative to detect at least one of speed and a trajectory of the applicator; and ii) the vibration generation device includes a vibration controller operative to provide at least one of a vibration frequency and a vibration amplitude in accordance with results of at least one of the speed detecting and the trajectory detecting.

According to some embodiments, the vibration controller is operative to provide in increased frequency contingent on detecting an increased applicator speed.

According to some embodiments, i) the apparatus further comprises a pulse width modulation controller operative to cause the RF power source to deliver the RF output signal in pulses of a given duration at a given repetition rate; and ii) the vibration generation device is operative to provide the vibration of the at least a portion at a mechanical vibration frequency determined in accordance with the RF pulse repetition rate.

According to some embodiments, the vibration generation device is operative such that the a ratio between the mechanical vibration frequency and the RF pulse repetition rate is one of:

i) an integer; and ii) a reciprocal of an integer.

According to some embodiments, the vibration generation device and the applicator are operative to provide maximum compression at times that are substantially a time of a RF pulse maximum of RF pulses.

According to some embodiments, the vibration mechanism includes: i) a motor; and ii) an eccentric weight mechanically coupled to the motor.

According to some embodiments, the vibration mechanism includes: i) a magnetic weight; and ii) one or more electromagnets operative to cause the magnetic weight to oscillate.

According to some embodiments, the vibration mechanism is operative to generate the mechanical vibrations of the at least a portion in a direction that is substantially perpendicular to a contact surface of the applicator.

According to some embodiments, the apparatus further comprises: d) a cooling device for cooling at least a portion of the biological tissue.

According to some embodiments, the apparatus lacks a cooling device.

According to some embodiments, the apparatus lacks a ground electrode for receiving electric current of the produced RF power.

According to some embodiments, the apparatus lacks a ground electrode for receiving electric current of the produced RF power.

It is now disclosed for the first time a method of treating biological tissue, the method comprising: a) delivering at least 10 Watts of RF power to the biological tissue from an applicator in contact with the biological tissue; b) concomitant with the RF power delivering, generating mechanical vibrations by a vibration generation device including vibrations having a frequency of at least 1 Hz and at most 100 Hz; and c) delivering the generated mechanical vibrations to the biological tissue.

According to some embodiments, the mechanical vibrations are delivered so as to repeatedly provide compression to the biological tissue at or beneath a contact interface between the applicator and the biological tissue at the frequency.

According to some embodiments, at least 10 consecutive cycles of the mechanical vibrations are delivered to the biological tissue. In different embodiments, at least 5 consecutive cycles, at least 15 consecutive cycles, at least 20 consecutive cycles, and at least 50 consecutive cycles are delivered.

According to some embodiments, at least 20 watts of the RF power is delivered to the biological tissue.

According to some embodiments, the method is performed for cellulite reduction.

According to some embodiments, the method is performed for collagen remodeling.

According to some embodiments, the method further comprises: c) controlling a phase of an electromagnetic wave carried by the delivered RF-power so that the delivered RF power is concentrated primarily in a predetermined energy dissipation zone, which lies at a desired depth beneath a surface of the biological tissue. According to some embodiments, the method further comprises: c) matching an impedance of a power source of the RF power with an impedance of the biological tissue.

According to some embodiments, the RF power delivery includes cyclically accumulating and releasing a desired amount of RF power.

According to some embodiments, the RF power is delivered to the biological tissue via a dielectric barrier.

According to some embodiments, mechanical vibrations of the biological tissue include vibrations in a direction that is substantially parallel to a wavefront propagation direction of the delivered RF power.

According to some embodiments, an average direction of the mechanical vibrations of the biological tissue caused by the vibration generation device is substantially parallel to a wavefront propagation direction of the delivered RF power.

According to some embodiments, the vibration generation device resides at least in part within the applicator.

According to some embodiments, the vibration generation device resides outside of the applicator.

According to some embodiments, the delivered mechanical vibrations have an amplitude of at least 0.1 mm. In different embodiments, the amplitude may be at least 0.2 mm, 0.3 mm, 0.5 mm or 1 mm.

According to some embodiments, an amplitude of the mechanical vibrations is at least 0.005 times a square root of a surface area of a contact interface between the applicator and the biological tissue.

According to some embodiments, the vibration generation device includes at least one of: i) a linearly oscillating mass; ii) a rotating eccentric weight; iii) an electromagnetic actuator; iv) a piezoelectric actuator; vi) a mangetostrictive actuator.

According to some embodiments, the method further comprises d) detecting a softness of the biological tissue; wherein at least one of a vibration frequency and a vibration amplitude of the delivered mechanical vibrations are determined in accordance with results of the tissue softness detecting. According to some embodiments, an increased the frequency is provided contingent on a detecting of an increased tissue softness.

According to some embodiments, the method further comprises d) detecting at least one of a speed and a trajectory of the applicator; wherein at least one of a vibration frequency and a vibration amplitude of the delivered mechanical vibrations are determined in accordance with results of at least one of the speed and the trajectory detecting.

According to some embodiments, i) the delivered RF power is pulsed RF power; and ii) at least one of an amplitude and a frequency of the delivered mechanical vibrations is determined in accordance with at least one pulse parameter of the pulsed RF power.

According to some embodiments, a ratio between a frequency of the delivered mechanical vibrations and a RF pulse repetition rate of the RF power is one of: i) an integer; and ii) a reciprocal of an integer.

According to some embodiments, the generated mechanical vibrations are delivered so as to provide maximum compressions at times that are substantially times of an RF pulse maximum of the pulsed RF power.

According to some embodiments, the method further comprises: d) cooling a surface of the biological tissue.

According to some embodiments, the method is carried out without cooling a surface of the biological tissue.

According to some embodiments, the delivered RF power is delivered from an apparatus lacking a ground electrode.

According to some embodiments, the delivered RF power is delivered from an apparatus having a ground electrode.

It is now disclosed for the first time a method of treating biological tissue, the method comprising: a) delivering at least 10 Watts of RF power to the biological tissue from an applicator in contact with the biological tissue; b) concomitant with the RF power delivering, using a vibration generation device, generating mechanical vibrations of at least a portion of the applicator including vibrations having a frequency of at least 1 Hz and at most 100 Hz.

These and further embodiments will be apparent from the detailed description and examples that follow.

### BRIEF DESCRIPTION OF THE DRAWINGS

FIGS. 1, 2A-2C and 4 illustrate exemplary systems for treating biological tissue with RF power using an applicator, at least a portion of which vibrates, according to some embodiments of the present invention.

FIGS. 3, 5-6 provide flow charts of exemplary techniques for treating biological tissue using an applicator that vibrates.

FIG. 7 provides a diagram of RF power and vibration amplitude as a function of time.

While the invention is described herein by way of example for several embodiments and illustrative drawings, those skilled in the art will recognize that the invention is not limited to the embodiments or drawings described. It should be understood that the drawings and detailed description thereto are not intended to limit the invention to the particular form disclosed, but on the contrary, the invention is to cover all modifications, equivalents and alternatives falling within the spirit and scope of the present invention. As used throughout this application, the word “may” is used in a permissive sense (i.e., meaning “having the potential to”), rather than the mandatory sense (i.e. meaning “must”).

### DETAILED DESCRIPTION OF EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the exemplary system only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice.

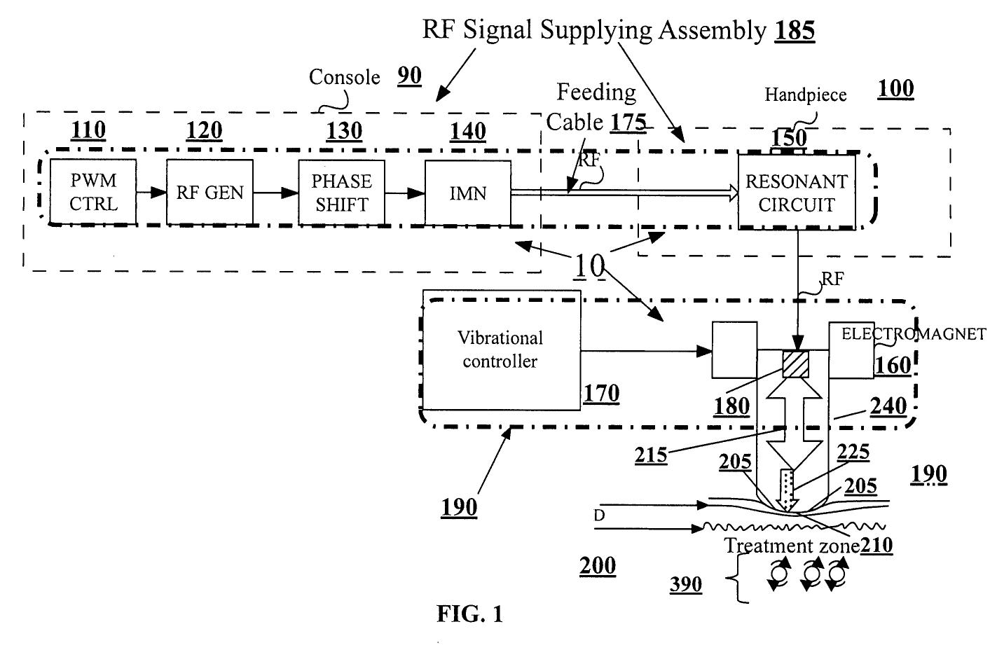

Reference is now made to FIG. 1.

### A Discussion of Components for Delivering RF Power to the Biological Tissue

The treatment apparatus 10 of FIG. 1 includes (i) an RF-signal supplying assembly 185 of electrical components for generating an RF output signal useful for delivery to biological tissue to heat the biological tissue; (ii) an RF applicator 240 or RF coupler (for example, attached to handpiece 100, or provided as a portion or an entirety of handpiece 100) for directing the RF signal to biological tissue in physical contact with biological tissue 200 such that RF-power is dissipated through the biological tissue; and (iii) a vibration generation device 190 operative to generate mechanical vibrations of at least a portion of the applicator/RF coupler 240—for example, mechanical vibrations of a lower surface 205 which is (A) in contact with an upper surface of the biological tissue 200 at a contact region 210 of the upper surface; (B) through which the generated RF signal is transmitted.

In the non-limiting example of FIG. 1, vibration generation device 190 includes an electromagnetic actuator that includes a linearly oscillating mass 180 set in motion by electromagnet assembly 160. The vibration generation device 190 further includes controller 170 for controlling one or more “vibration parameters” for example vibration amplitude and/or frequency.

Typically, the mechanical vibrations have a frequency of between 1 Hz and 100 Hz, and amplitude of between 0.1-10 mm.

In some embodiments, the mechanical vibrations the mechanical vibrations have a frequency of between 1 Hz and 10 Hz.

As noted above, treatment apparatus 10 of FIG. 1 includes a RF-signal supplying assembly 185 that provides an RF output signal delivered to biological tissue 200 by applicator/RF coupler 240. In the non-limiting example of FIG. 1, RF-signal providing assembly includes 185: (i) an RF generator 120 for generating RF power (in one non-limiting example, REF power having a frequency of at least 0.5 MHZ (megahertz) and less than 10 GHZ (gigahertz)); (ii) a phase shifter 130 operative to shift a phase of electromagnetic wave of the generated RF power; (ii) an impedance matching network (IMN) 140, operative to match an impedance of the output RF power generator into the biological tissue; and (iii) an RF resonator 150 connected to the applicator 240, the RF resonator operative to cyclically accumulate and release a desired amount of energy.

As discussed in US 2007/0106349, in some embodiments, phase shifter 130 is useful for shifting a phase of directed traveling waves of the output RF signal so that RF power is delivered “deeper layers” of treatment. Thus, in some embodiments, phase shifter 130 is provided to alter the RF output signal phase so that energy in concentrated at a predetermined zone at a desired depth beneath the surface of biological tissue.

Furthermore, in some embodiments, RF-signal supplying assembly 185 includes a feeding half-wave cable, for example, as discussed in US 2007/0106349.

In some embodiments, IMN 140 is operative to match the impedance of biological tissue 200 from a nominal value (e.g. 250-350 Ohms) to a corrected value, for example, an output impedance of RF-generator (e.g. 50 Ohms). The corrected value matches an impedance characteristic of RF energy generator 120 and RF transmission line including phase shifter 130 and feeding cable 175 so that reflection power from the treating tissue is minimal.

In the particular example of FIG. 1, pulse-modulated RF power is delivered, and the RF-signal supplying assembly 185 also includes a pulse width modulation controller 110, operative to causing the RF energy generator 120 to deliver the RF power in pulses of a predetermined duration and amplitude with a desired frequency.

The apparatus in FIG. 1 is configured to deliver RF power to a pre-determined treatment zone 390 beneath the surface of the biological tissue. In one example, the tissue in the treatment zone 390 is heated by electromagnetically inducing rotations of water dipoles.

It is appreciated that RF-signal supplying assembly 185 is not required, in every embodiment, to include every component in FIG. 1.

There is no limitation on the dimensions of applicator 240. In one non-limiting example, the applicator diameter is from 5 to 25 mm, for example between 10 and 18 mm.

In the example of FIG. 1, the treatment apparatus 10 is a so-called unipolar device which lacks a return ground electrode and where the treated biological tissue 200 thus functions as an antenna. The absence of a second or ground electrode in the pictured configuration permits free propagation of RF waves inside tissue 200.

Thus, in some preferred embodiments, the provided treatment apparatus 10 is a unipolar device where applicator 240 functions as a single device electrode or electromagnetic “coupler” in physical or capacitive contact with and radiatively coupled with the biological tissue 200.

In alternate embodiments, treatment apparatus 10 is a so-called “bipolar” devices (i.e. including vibration generation device 190) for delivering RF power to biological tissue that feature a ground plane electrode (not shown).

As noted, in some embodiments, RF-signal supplying assembly 185 includes pulse width modulator controller 110, capable of causing the RF energy source to deliver the output signal in pulses of a desired amplitude, a predetermined duration with a desired repetition frequency for average output power control. One exemplary pulse width modulator controller 110 is described in US 2007/0106349.

In one particular example, 25-300 watts of power are delivered, the operating RF-frequency is 40.68 MHz, a PWM-frequency is 0.5 to 50 kHz and a duty cycle is 1 to 100%.

Specifically, in these embodiments, control of phase and pulse with modulation (PWM)-control of applied RF waves through conductive applicator 240 which functions as or includes “a single electrode” may obviate the need for cooling of the skin surface while facilitating efficient heating of underlying layers of tissue such as dermis and subcutaneous layers.

Application of high RF-power in short-pulses may provide fast and effective heating of cellulite capsules with relatively low average RF-power level.

In some embodiments, treatment apparatus 10 includes a cooling element for cooling a skin surface.

### A Discussion of Applicator 240, Vibration Direction Vector 215, and RF Wavefront Propagation Vector 225

In the present section, it is disclosed that in some embodiments, it is advantageous to have at least a portion of applicator 240 (typically a lower “contact” surface 205) mechanically vibrate in a direction substantially (for example, within a tolerance of 45 or 30 or 15 degrees) “perpendicular” to an upper surface of tissue 200.

As illustrated in the figures, both applicator 240 and resonator 150 are associated with handpiece 100. In one use scenario, a user (for example, a medical professional administering the RF energy) moves handpiece 100 including applicator 240 over the surface of the biological tissue 200 to treat the tissue. When “applicator contact region” 205 of the lower surface of applicator 240 contacts a “tissue surface contact region” 210 of the upper surface of the biological tissue 200, RF power is delivered from the applicator 240 to the biological tissue 200. As shown in FIG. 1, applicator 240 delivers RF power to the biological tissue through the “contact surface” between applicator contact region 205 and tissue surface contact region 210 or the “contact interface”. RF Wavefront Propagation Vector 225 represents a direction of propagation of a wavefront of the delivered RF power that is delivered from the applicator 240 to the biological tissue 200.

As shown in FIG. 1, vibration direction vector 215 represents the average direction of vibration of “applicator contact region” 205 of the lower surface of applicator 240. In the example of FIG. 1, vibration direction vector 215 is parallel to RF Wavefront Propagation Vector 225. In some embodiments, vibration direction vector 215 is “substantially parallel to” RF Wavefront Propagation Vector 225—i.e. parallel within a given tolerance—for example, within 45 degrees, or within 30 degrees, or within 15 degrees, or within 5 degrees.

It is noted that each location within the “contacting region 210” of the upper surface of biological tissue (i.e. the portion of the upper surface in contact with applicator 240) may be associated with a “local surface vector” perpendicular to the local plane at the contacting location. The entirety of the “contacting region 210” of the upper surface of the biological tissue may be associated with a “contacted surface vector” (not shown) that is the average of all of the aforementioned local surface vectors. In the example of FIG. 1, vibration direction vector 215 is thus “substantially parallel to” to the contacted surface vector—i.e. parallel within a given tolerance for example, within 45 degrees, or within 30 degrees, or within 15 degrees, or within 5 degrees.

In embodiments where “vibration direction vector” 215 is substantially parallel to the contacted surface vector and/or RF Wavefront Propagation Vector 225, it is noted that the vibration may be useful for alternatively compressing the biological tissue 200 and allowing the biological tissue to “relax” or return to its “uncompressed form.”

The present inventor is disclosing that this may be useful when concomitantly treating the biological tissue with RE power to heat the biological tissue 200.

### Dielectric Material 220

As noted earlier, in different embodiments, the treatment apparatus 10 may include any combination of one or more features disclosed in US 2007/0106349. Thus, it is noted that in various embodiments, applicator 240 (i) is made primarily from “conductive” materials (for example, having an electrical conductivity that exceed a conductivity of iron, or that exceeds a conductivity of nickel, or that exceeds a conductivity of tungsten) for example one or more metals including but not limited to Al, Ag, Au, copper, and/or alloys thereof and (ii) is associated with a dielectric material 220 that serves as a barrier between the conductive applicator and the biological tissue. In one non-limiting example, the dielectric material 220 is provided as a coating to the conductive material of applicator 240.

### A Discussion of Vibration Generation Device 190: Several Example Implementations

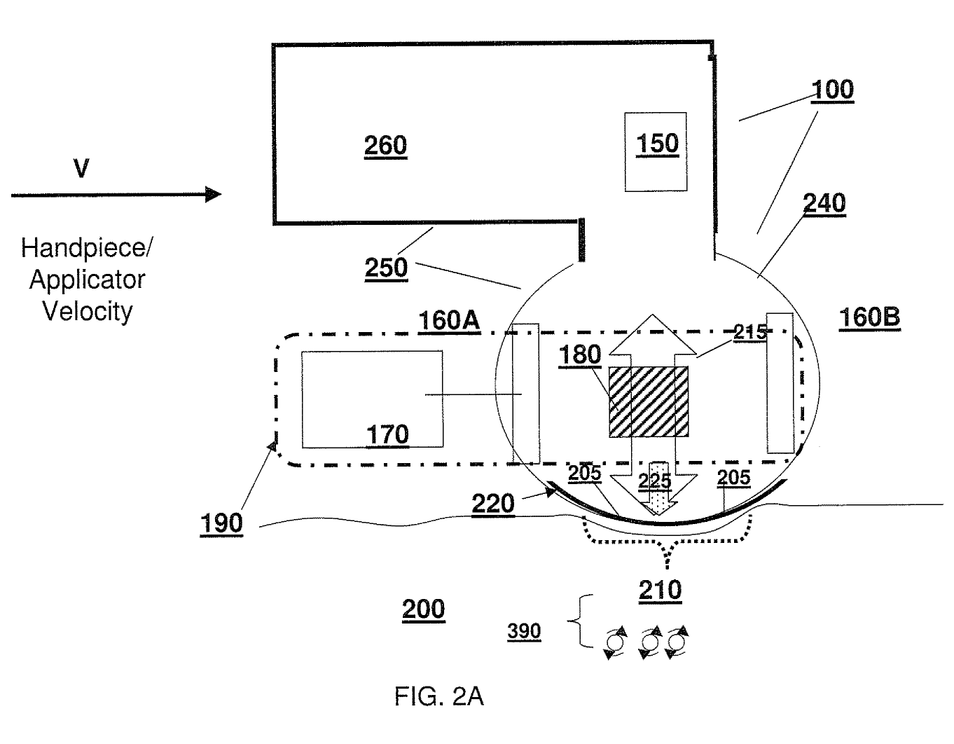

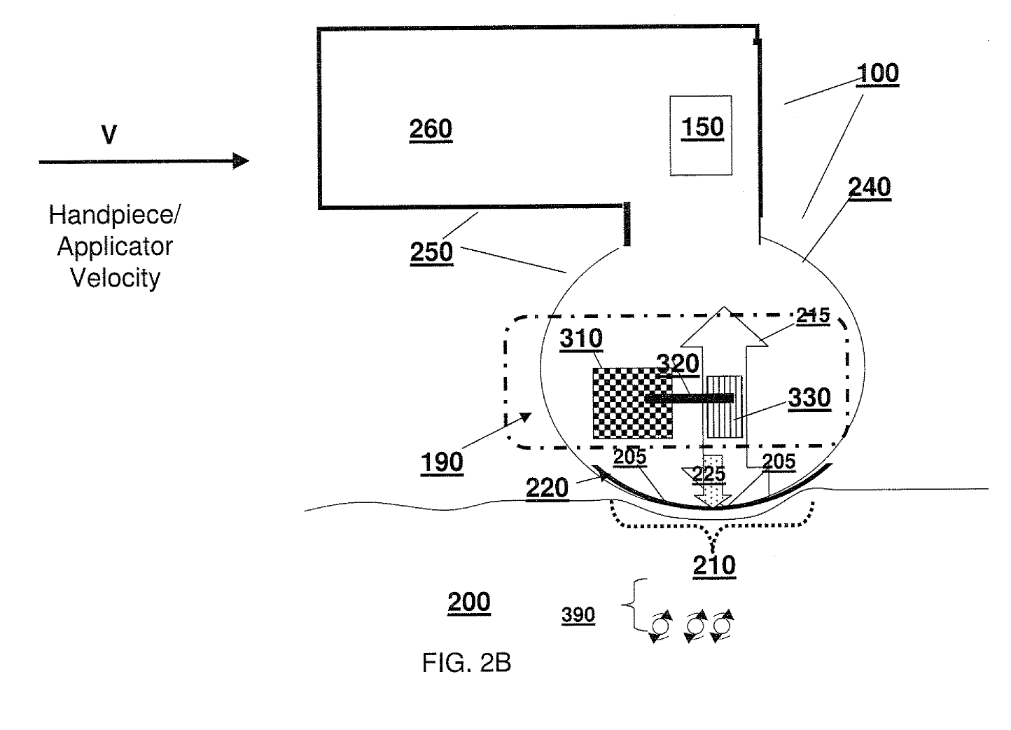

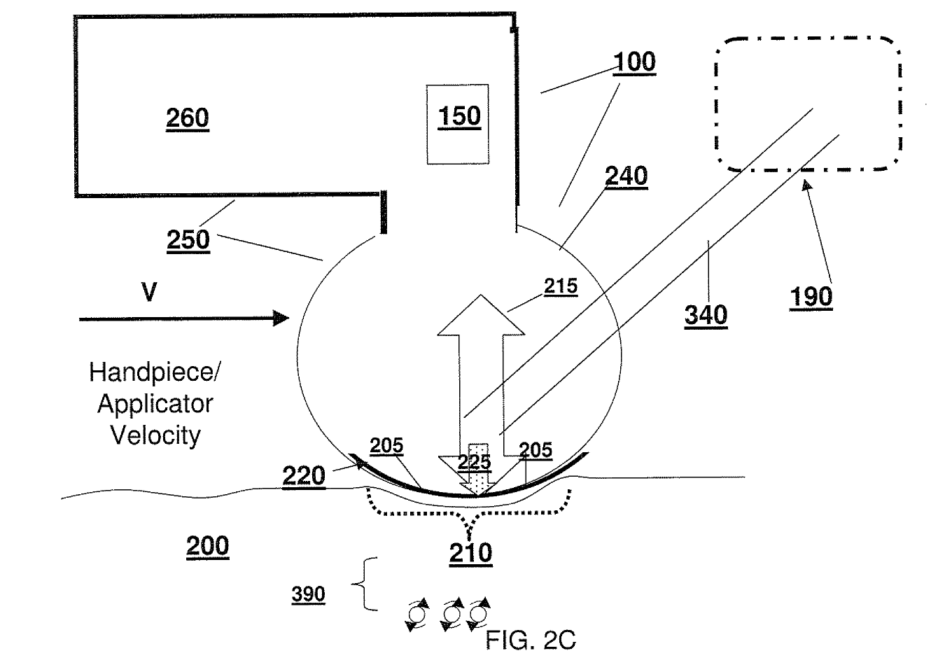

FIGS. 2A-2C provide diagrams of a handpiece 100 including an applicator 240 (i.e. at least a portion of which mechanically vibrates) associated with handpiece 100. In the example of FIG. 2A, applicator 240 associated with handpiece 100 moves over the surface of biological tissue 200 with a velocity v.

In the examples of FIGS. 1 and 2A, vibration generation device 190 includes an electromagnetic actuator (i.e. including electromagnet(s) 160 and linearly oscillating mass 180). It is noted that this is just one exemplary limitation, and that vibration generation device 190 may include any structure for causing at least a portion of applicator 240 to mechanically vibrate.

In some embodiments, certain teachings of U.S. Pat. No. 6,481,104 and U.S. Pat. No. 5,299,354, incorporated herein by reference, are adopted for the vibration generation device 190.

In FIG. 2B, vibration generation device 190 includes a rotary motor 310 (for example, a DC motor, an AC motor or any other type of motor) connected to an eccentric element 330 via shaft 320. Rotation of shaft 320 causes eccentric weight 330 to move within applicator 240, and to impart vibratory motion on at least a portion of applicator 240.

In the example of FIG. 2B, Vibration generation device 190 resides within applicator 240—in the example of FIG. 2B, within a hollow portion of applicator 240. Nevertheless, this should not be construed as a limitation. In alternate embodiments, one or more components of vibration generation device 190 may reside outside of applicator 240, for example, mounted to applicator 240, or even “remotely” as illustrated in FIG. 2C, where vibrations from a vibration generation element 190 are “transmitted” via a vibration transmitter 340 (for example, implemented hydraulically or pneumatically) for transmitting “remotely generated” vibrations to applicator 240.

It is appreciated that these are merely a few examples, and that other vibration generation device 190 configurations (i.e. other than those explicitly illustrated in the present examples) for generating vibrations to impart mechanical vibrations to at least a portion of applicator 240 may be used.

In one non-limiting example, vibration generation device 190 includes a piezoelectric device and/or magnetostrictive actuator to provide mechanical vibrations.

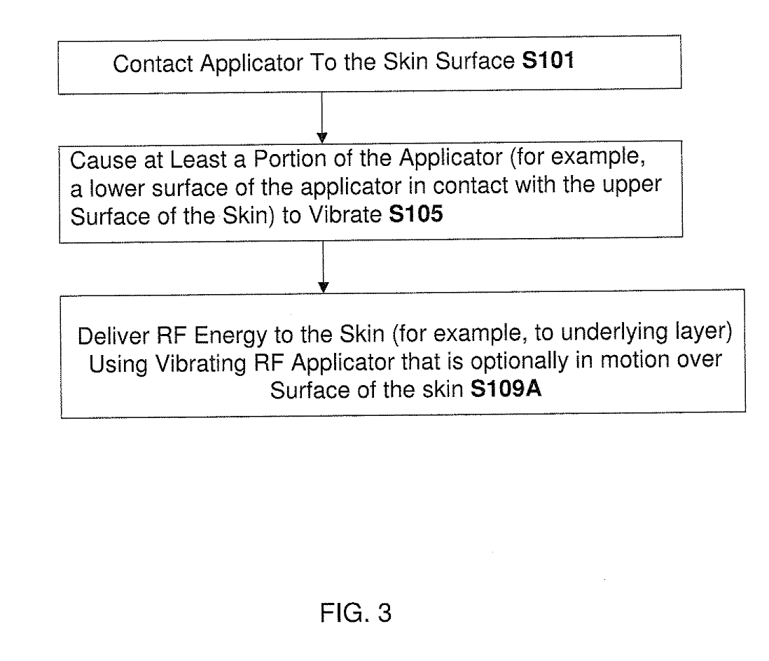

### A Discussion of FIG. 3

FIG. 3 provides a flow diagram of an exemplary technique for treating biological tissue 200 using an applicator 240, at least a portion of which mechanically vibrates. In step S101, the applicator 240 is contacted to the upper surface of biological tissue 200. In step S105, at least a portion of applicator 240 is caused to vibrate (for example, in a direction substantially “perpendicular” to an upper surface of tissue 200).

In step S109A, RF energy is delivered to the biological tissue at a time at least a portion of RF applicator 240 vibrates.

It is noted for all “flow diagrams” provided herein that although the steps may be carried out in the order specified (for example, the vibrations of S105 may of course commence before contact between applicator 240 and biological tissue 200 is established), this is certainly not a requirement.

### Vibration Parameters

In some embodiments, vibration generation device 190 is operative to cause at least a portion of applicator (for example, a lower surface 205 in contact with an upper surface 210 of the biological tissue 200) to vibrate with an amplitude of between 0.2 and 6 mm. It is also appreciated that different vibration frequencies (i.e. for vibration of at least a portion of applicator 240 and/or provided by vibration generation device 190) may be used in different embodiments. In some embodiments, the frequency is between 1 Hz and 100 Hz. In some embodiments, the frequency is between 2 Hz and 10 Hz.

It is appreciated that the amplitude may vary between individual vibration cycles, and is not necessarily constant. Additionally, it is appreciated that the “vibration frequency” is not required to be constant and may vary between vibration cycles.

In some embodiments, the vibration parameters are “fixed” and/or “hardwired.” Alternatively, the vibration parameters may be provided by a user of the apparatus 10 (for example, a medical professional) and/or may be provided by the apparatus 10 itself (or a component thereof) in response to one or more detected parameters.

### A Discussion of Different Use Scenarios Where a Treatment-Administrator Manually Provides One or More Vibration Parameters

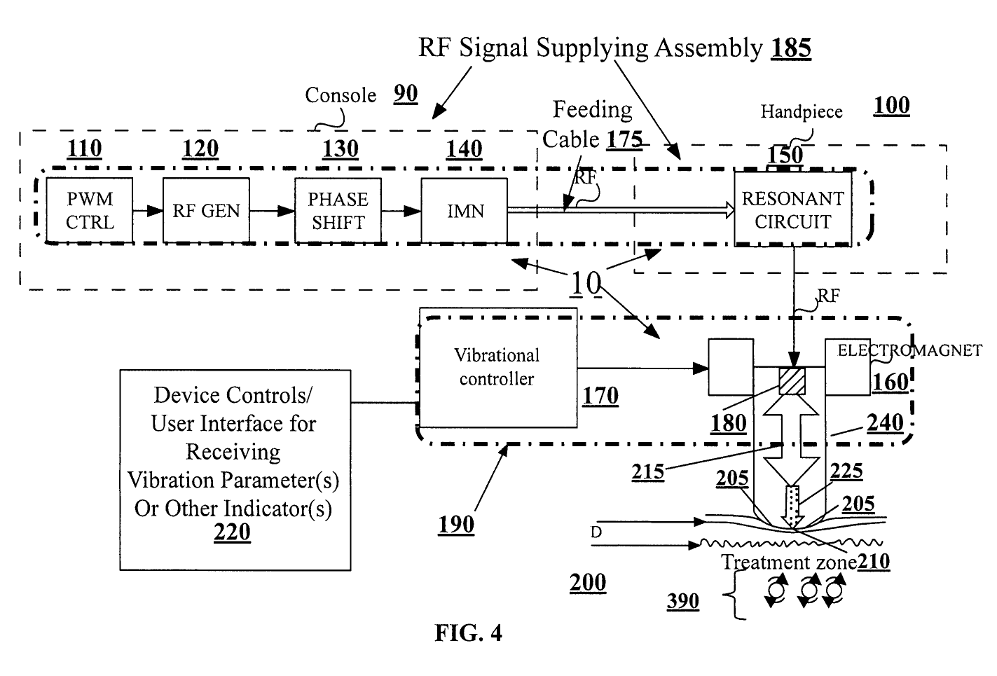

Thus, in the example FIG. 4, the apparatus 10 includes device controls/user interface 220 (e.g. mechanical or electrical or electronic for example, including one or more buttons or dials or other user controls) for receiving one or more vibration parameters. User interface 220 is operatively linked to vibration controller 170, and in the example of FIG. 3, vibration controller 170 is operative to set one or more vibration parameters according to user information received via user interface 190.

According a first use scenario, causing at least a portion of applicator 240 to vibrate may be useful, for example, when it is desired to facilitate contact between a lower surface of applicator 240 and an upper surface of biological tissue 205.

Thus one example relates to “soft tissue” which may have a tendency to “move” during treatment. The present inventor notes that, the likelihood of “losing contact” (i.e. touch and capacitive coupling) when treating softer tissue may be greater than the likelihood of “loosing contact” when treating harder tissue.

The present inventor is now disclosing that mechanical vibrations of at least a portion of applicator 240 (such as a lower “contact” surface 205) are useful for increases the probability of contact during a time period when contact may otherwise be lost by a “rule of averages”—i.e. since the instantaneous location (i.e. for example, in the “z” direction perpendicular to the local surface of the biological tissue) of the lower surface 205 of applicator 240 changes in time due to the vibrations, on average the probability that the lower surface 205 of applicator 240 would be in the “right location” for contacting tissue 240 at least “some” of the time would increase due to the mechanical vibrations.

Thus, according to a first use scenario, the user (e.g. for example, medical practitioner administering the RF treatment to the subject of the biological tissue) may (i) note that s/he is to treat “soft tissue”; and (ii) input, via user interface 220 (or “device controls”), a set of vibration parameters that includes a “higher” mechanical vibration frequency.

According to a second use scenario, the present inventor is noting that as the speed v of applicator 240 over the surface of biological tissue increases, it is possible that the probability of “losing capacitive contact” between a lower surface 205 of applicator 240 and biological tissue 200 may increase due to applicator speed. Thus, according to this scenario, the user or RF treatment administrator may select, via device controls 220, a higher frequency when s/he intends to use a “higher” applicator 240 speed.

Alternatively or additionally, one or more vibration parameters may be provided “implicitly.” Thus, in some embodiments, the user interface 220 is operative to receive (i) information such as handpiece moving speed (or alternatively, a selection from a pre-determined list such as “slow speed,” “medium speed,” and “fast speed”); and/or (ii) information such as tissue softness; and/or (iii) any other information. In accordance to the information received via user interface 220, one or more vibration parameters (for example, frequency or amplitude) may be computed.

A Discussion of Several Routines for “Automatically” or “Adaptively” Selecting and/or Adjusting Vibration Parameters in Response to One or More Detected Physical Parameters

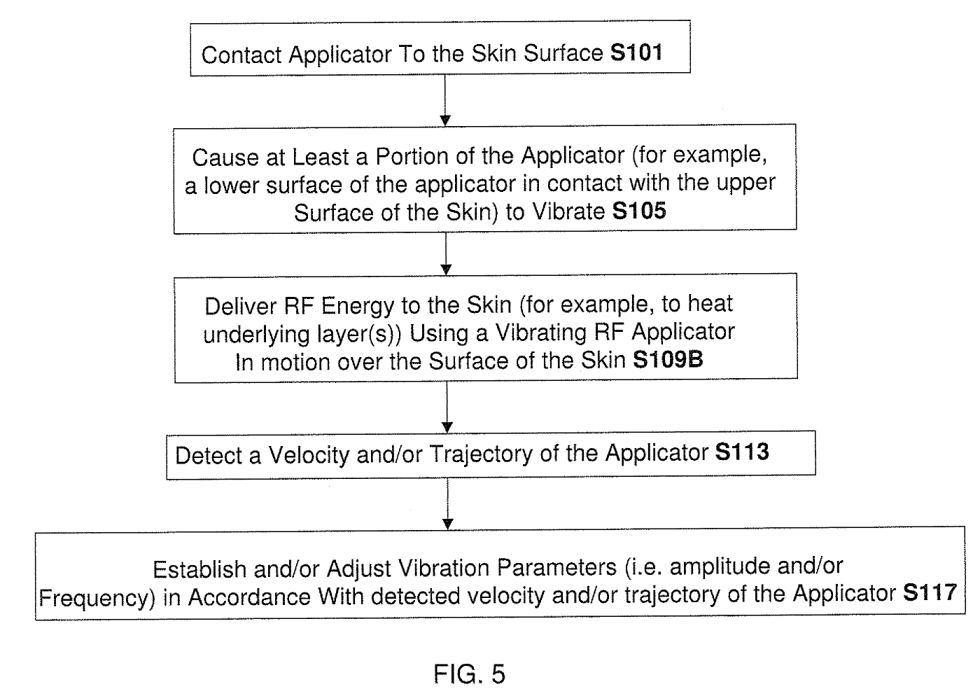

FIG. 5 provides a flow diagram of a routine for treating biological tissue with RF energy according to some embodiments. In the example of FIG. 5, the vibration frequency is determined in accordance with the applicator 240 velocity.

In FIG. 5, steps S101 and S105 are as in FIG. 3. In step S109B, RF energy is delivered from applicator 240 to biological tissue when applicator 240 is in motion over the biological tissue.

In step S113, a velocity and/or trajectory of the applicator 240 is detected. Any known technique or apparatus for determining applicator 240 position or velocity (mechanical, electrical or otherwise) may be used.

In one non-limiting example, the position of applicator 240 is detected using ultrasound “triangulation” position detecting system. For example, the applicator 240 may be associated with or connected to or include an ultrasound transmitter and an IR transmitter. In addition, two or more ultrasound receivers (i.e. whose position is fixed in space) and one or more IR receiver may be provided. Using the IR signal for synchronization, time or flight data for two or more ultrasound receivers may be useful for providing the location of applicator 240 at any given time. Velocity and/or trajectory data may derive from the position data as a function of time. It is, once again, noted that this is merely a non-limiting example.

In step S117, one or more vibration parameter(s) are established and/or adjusted in accordance with the detected velocity and/or trajectory of applicator 240. In one example, in response to a “faster” handpiece velocity, increased frequency and/or amplitudes are provided.

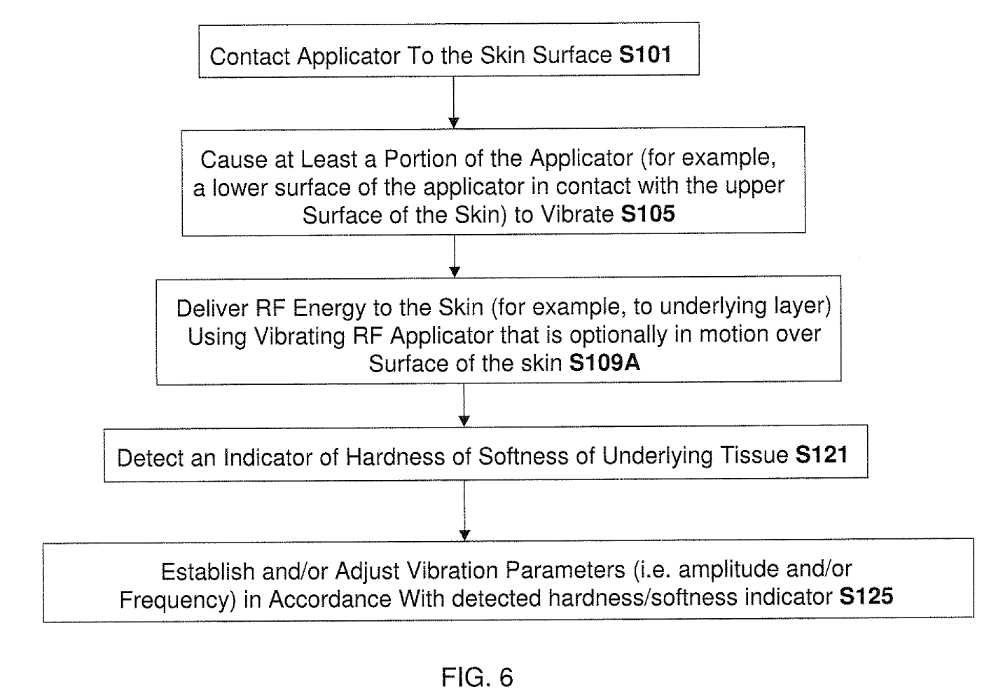

FIG. 6 provides a flow diagram of a routine for treating biological tissue with RF energy according to some embodiments. In the example of FIG. 6, the vibration frequency is determined in accordance with the sensed tissue “softness.”

In FIG. 6, steps S101, S105, and S109B are as in FIG. 3. In step S121 an indication of tissue hardness or softness is sensed. Any known technique or apparatus for determining tissue softness (mechanical, electrical or otherwise) may be used.

In one non-limiting example, a speed of sound is measured through the tissue is measured and this correlates with tissue softness.

In another non-limiting example, vibrations of a pre-determined amplitude are provided (for example, when a lower surface 205 of applicator 240 is in good contact with an upper surface, and the amount of current required to provide these vibrations is measured. In the event that the tissue is “hard tissue” more current would be consumed, and if the tissue is “soft” tissue less current would be consumed.

In step S125, one or more vibration parameter(s) are established and/or adjusted in accordance with the detected tissue softness. In one example, in response to a “faster” handpiece velocity, increased frequency and/or amplitudes are provided.

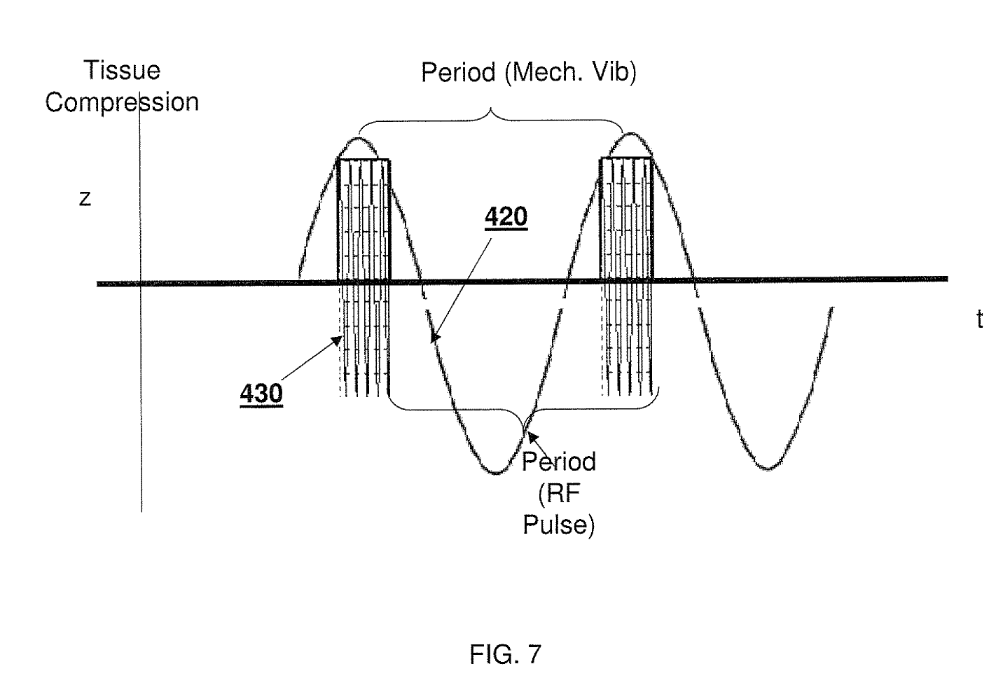

A Discussion of FIG. 7-Providing Mechanical Vibrations “in Phased with” or “Synchronized with” RF Pulses

The present inventor is now disclosing, that in some situations related to delivering pulsed RF power having a “pulse frequency”, it may be advantageous to provide mechanical vibrations with a frequency that is substantially equal to (within a given tolerance, such at 10% or 5% or 1% or 0.5%) an integral multiple (or a reciprocal of an integral multiple) of the RF pulse frequency. In some embodiments, it is possible to “synchronize” the mechanical vibrations with the RF pulses.

In one example where the direction of the mechanical vibrations is substantially perpendicular to a local upper surface of the biological tissue 200, it may be advantageous to do this such that the lower surface 205 of applicator 240 is at its “maximal low point” (i.e. providing maximum compression of biological tissue 200) at a time that the RF pulse amplitude is maximum. In some embodiments, the maximums of the mechanical vibration amplitude and the RF pulse amplitude are thus substantially “synchronized”—i.e. occur at the same time within a tolerance that is at most, for example, at most 10% or 5% or 1% or 0.5% of a shorter “period” (i.e. frequency reciprocal)—i.e. the shorter “period” of the RF power or the mechanical vibration.

The present inventor is disclosing that providing maximum compression at a time of maximum RF power may useful, for example, for (i) increasing the probability that the lower surface 205 is in contact with the upper surface 210 of biological tissue at the “most important” moment in time—i.e. when the RF pulse is at its maximum intensity; and (ii) may be useful for providing a “synergy” effect between tissue compression and administration of RF energy. In one example related to treating “deeper” layers of tissue, providing this compression may be useful for shortening, in absolute terms, the distance between the lower surface 205 of applicator 240 and the target deeper layers of tissue 210.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

All references cited herein are incorporated by reference in their entirety. Citation of a reference does not constitute an admission that the reference is prior art.

The articles “a” and “an” are used herein to refer to one or to more than one (i.e., to at least one) of the grammatical object of the article. By way of example, “an element” means one element or more than one element.

The term “including” is used herein to mean, and is used interchangeably with, the phrase “including but not limited” to.

The term “or” is used herein to mean, and is used interchangeably with, the term “and/or,” unless context clearly indicates otherwise.

The term “such as” is used herein to mean, and is used interchangeably, with the phrase “such as but not limited to”.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons of the art.

What is claimed is:

1) An apparatus for treatment of a biological tissue of a subject comprising:
a) an applicator 240 contactable with a surface 210 of the tissue;
b) an RF power source 120 configured to produce at least 20 Watts of RF power directed to said applicator 240; and
c) a vibration generation device 190 mechanically linked to said applicator 240, said vibration generation device 190 being operative to generate mechanical vibrations of at least a portion 205 of said applicator 240 including mechanical vibrations having a frequency of at least 1 Hz and at most 100 Hz.

2) The apparatus of claim 1 further comprising:
a phase shifter 130 operative to control a phase of electromagnetic wave carried the said RF-power.

3) The apparatus of claim 1 further comprising:
an impedance matching network (IMN) 140, operative to match an impedance power source to impedance of the biological tissue.

4) The apparatus of claim 1 further comprising:
an RF resonator 150 connected to said applicator, said RF resonator operative to cyclically accumulate and release a desired amount of RF energy.

5) The apparatus of claim 1 wherein said applicator includes only a single electrode with a dielectric barrier associated with an outside surface of said applicator.

6) The apparatus of any of claim 1 wherein said applicator is made primarily from electrically conductive materials.

7) The apparatus of claim 1 wherein said RF power source 120, said applicator 240 and said vibration generation device 190 are configured so that, when said applicator 240 is contacted to the surface of the biological tissue:
i) said 240 applicator is operative to deliver said RF power to the contacted biological tissue;
ii) said vibration generation device 190 is operative such that said mechanical vibrations of said at least a portion of said applicator 240 include vibrations in a direction that is substantially parallel to a wavefront propagation direction 225 of said RF power delivered from said applicator 240 to the biological tissue.

8) The apparatus of claim 1 wherein said RF power source 120, said applicator 240 and said vibration generation device 190 are configured so that, when said applicator 240 is contacted to the surface of the biological tissue:
i) said 240 applicator is operative to deliver said RF power to the contacted biological tissue via an applicator contact region 205 of the applicator;
ii) said vibration generation device 190 and said applicator 240 are operative such that an average direction of generated mechanical vibrations at said applicator contact region 205 is substantially parallel to a wavefront propagation direction 225 of said RF power delivered from said applicator 240 to the biological tissue.

9) The apparatus of claim 1 wherein said vibration generation device 190 and said applicator 240 are configured such that said generated mechanical vibrations of said at least a portion include mechanical vibrations having a frequency of at least 2 Hz and at most 10 Hz.

10) The apparatus of claim 1 wherein said vibration generation device 190 and said applicator 240 are configured such that said generated mechanical vibrations of said at least a portion include mechanical vibrations having an amplitude of at least 0.1 mm and more.

11) The apparatus of claim 1 wherein said vibration generation device 190 and said applicator 240 are configured such that said generated mechanical vibrations of said at least a portion include mechanical vibrations having an amplitude of at most 10 mm.

12) The apparatus of claim 1 wherein said vibration generation device 190 includes a linearly oscillating mass 180.

13) The apparatus of claim 1 wherein said vibration generation device 190 includes:
i) a rotary motor 310; and
ii) a rotary-to-linear motion (320, 330) converter operatively linked to said motor.

14) The apparatus of claim 1 wherein said vibration generation device 190 is embedded within said applicator 240.

15) The apparatus of claim 1 wherein said vibration generation device 190 is operative to generate remote vibrations remote to said applicator 240, the apparatus further comprising:
a vibration transmitter 340 operative to transmit said remote vibration to said applicator

16) The apparatus of claim 1 wherein said vibration generation device includes at least one of:
i) an electromagnetic actuator (160, 180);
ii) a piezoelectric actuator; and
iii) a magnetostrictive actuator.

17) The apparatus of any of claim 1 wherein:
i) the apparatus further comprises a tissue softness detector operative to detect S121 a softness of the biological tissue contacted by said applicator 240; and
ii) said vibration generation device 190 includes a vibration controller 170 operative to provide S125 at least one of a vibration frequency and a vibration amplitude in accordance with results of said tissue softness detecting.

18) The apparatus of claim 17 wherein said vibration controller is operative to provide in increased frequency contingent on detecting increased tissue softness.

19) The apparatus of claim 1 wherein:
i) the apparatus further comprises a applicator movement speed detector operative to detect S113 at least one of speed and a trajectory of said applicator 240; and
ii) said vibration generation device 190 includes a vibration controller 170 operative to provide S117 at least one of a vibration frequency and a vibration amplitude in accordance with results of at least one of said speed detecting and said trajectory detecting.

20) The apparatus of claim 19 wherein said vibration controller is operative to provide in increased frequency contingent on detecting an increased applicator speed.

21) The apparatus of claim 1 wherein:
i) the apparatus further comprises a pulse width modulation controller 110 operative to cause said RF power source to deliver said RF output signal in pulses of a given duration at a given repetition rate; and
ii) said vibration generation device 190 is operative to provide said vibration of said at least a portion at a mechanical vibration frequency determined in accordance with said RF pulse repetition rate.

22) The apparatus of claim 21 wherein said Vibration generation device 190 is operative such that said a ratio between said mechanical vibration frequency and said RF pulse repetition rate is one of:
i) an integer; and
ii) a reciprocal of an integer

23) The apparatus of claim 21 wherein said vibration generation device 190 and said applicator are operative to provide maximum compression at times that are substantially a time of a RF pulse maximum of RF pulses.

24) The apparatus of claim 1 wherein said vibration mechanism 190 includes:
i) a motor 310; and
ii) an eccentric weight 330 mechanically coupled to said motor.

25) The apparatus of claim 1 wherein said vibration mechanism 190 includes:
i) a magnetic weight 180; and
ii) one or more electromagnets 160 operative to cause said magnetic weight to oscillate.

26) The apparatus of claim 1 wherein said vibration mechanism 190 is operative to generate said mechanical vibrations of said at least a portion in a direction that is substantially perpendicular to a contact surface 205 of said applicator 240.

27) The apparatus of claim 1 further comprising:
d) a cooling device for cooling at least a portion of the biological tissue.

28) The apparatus of claim 1 wherein the apparatus lacks a cooling device.

29) The apparatus of claim 1 wherein the apparatus lacks a ground electrode for receiving electric current of said produced RF power.

30) The apparatus of claim 1 wherein the apparatus lacks a ground electrode for receiving electric current of said produced RF power.

31) A method of treating biological tissue, the method comprising:
a) delivering at least 10 Watts of RF power to the biological tissue from an applicator 240 in contact with the biological tissue,
b) concomitant with said RF power delivering, generating mechanical vibrations by a vibration generation device 190 including vibrations having a frequency of at least 1 Hz and at most 100 Hz; and
c) delivering said generated mechanical vibrations to the biological tissue.

32) The method of claim 31 wherein said mechanical vibrations are delivered so as to repeatedly provide compression to the biological tissue at or beneath a contact interface 210 between said applicator and the biological tissue at said frequency.

33) The method of claim 31 wherein at least 10 consecutive cycles of said mechanical vibrations are delivered to the biological tissue.

34) The method of claim 31 wherein at least 20 watts of said RF power is delivered to the biological tissue.

35) The method of claim 31 wherein the method is performed for cellulite reduction.

36) The method of claim 31 wherein the method is performed for collagen remodeling.

37) The method of claim 31 further comprising:
c) controlling a phase of an electromagnetic wave carried by said delivered RF-power so that said delivered RF power is concentrated primarily in a predetermined energy dissipation zone, which lies at a desired depth beneath a surface of the biological tissue.

38) The method of claim 31 further comprising:
c) matching an impedance of a power source of said RF power with an impedance of the biological tissue.

39) The method of claim 31 wherein said RF power delivery includes cyclically accumulating and releasing a desired amount of RF power.

40) The method of claim 31 wherein said RF power is delivered to the biological tissue via a dielectric barrier.

41) The method of claim 31 wherein said mechanical vibrations of the biological tissue include vibrations in a direction that is substantially parallel to a wavefront propagation direction 225 of said delivered RF power.

42) The method of claim 31 wherein an average direction 215 of said mechanical vibrations of the biological tissue caused by said vibration generation device 190 is substantially parallel to a wavefront propagation direction 225 of said delivered RF power.

43) The method of claim 31 wherein said vibration generation device 190 resides at least in part within said applicator 240.

44) The method of claim 31 wherein said vibration generation device 190 resides outside of said applicator 240.

45) The method of claim 31 wherein said delivered mechanical vibrations have an amplitude of at least 0.1 mm.

46) The method of claim 31 wherein an amplitude of said mechanical vibrations is at least 0.005 times a square root of a surface area of a contact interface 210 between said applicator and the biological tissue.

47) The method of claim 31 wherein said vibration generation device includes at least one of:
i) a linearly oscillating mass;
ii) a rotating eccentric weight;
iii) an electromagnetic actuator;
iv) a piezoelectric actuator;
v) a mangetostrictive actuator.

48) The method of claim 31 further comprising:
d) detecting S121 a softness of the biological tissue;
wherein at least one of a vibration frequency and a vibration amplitude of said delivered mechanical vibrations are determined in accordance with results of said tissue softness detecting.

49) The method of claim 31 wherein an increased said frequency is provided contingent on a detecting of an increased tissue softness.

50) The method of claim 31 further comprising
d) detecting S113 at least one of a speed and a trajectory of said applicator 240;
wherein at least one of a vibration frequency and a vibration amplitude of said delivered mechanical vibrations are determined in accordance with results of at least one of said speed and said trajectory detecting.

51) The method of claim 31 wherein:
i) said delivered RF power is pulsed RF power, and
ii) at least one of an amplitude and a frequency of said delivered mechanical vibrations is determined in accordance with at least one pulse parameter of said pulsed RF power.

52) The method of claim 31 wherein a ratio between a frequency of said delivered mechanical vibrations and a RF pulse repetition rate of said RF power is one of:
i) an integer; and
ii a reciprocal of an integer.

53) The method of claim 31 wherein said generated mechanical vibrations are delivered so as to provide maximum compressions at times that are substantially times of an RF pulse maximum of said pulsed RF power.

54) The method of claim 31 further comprising:
d) cooling a surface of said biological tissue.

55) The method of claim 31 wherein the method is carried out without cooling a surface of the biological tissue.

56) The method of claim 31 wherein said delivered RF power is delivered from an apparatus lacking a ground electrode.

57) The method of claim 31 wherein said delivered RF power is delivered from an apparatus having a ground electrode.

58) A method of treating biological tissue, the method comprising:
a) delivering at least 10 Watts of RF power to the biological tissue from an applicator in contact with the biological tissue;
b) concomitant with said RF power delivering, using a vibration generation device, generating mechanical vibrations of at least a portion of said applicator including vibrations having a frequency of at least 1 Hz and at most 100 Hz.
